(19) United States
(12) Patent Application Publication
(10) Pub. No.: US 2006/0128579 A1
(43) Pub. Date: Jun. 15, 2006

(54) Cleansing foaming lotion

(76) Inventors: Michael A. Knopf, Randolph, NJ (US); Michele Polk, Flanders, NJ (US); Frank A. Lucia III, Wantage, NJ (US); William C. Wohland, Succasunna, NJ (US); Ralph Macchio, Sparta, NJ (US)

Correspondence Address: Schwegman, Lundberg, Woessner & Kluth, P.A., P.O. Box 2938, Minneapolis, MN, 55402, US

(21) Appl. No.: 11/176,697

(22) Filed: Jul. 7, 2005

Related U.S. Application Data
(63) Continuation-in-part of application No. 10/710,052, filed on Jun. 15, 2004.
(60) Provisional application No. 60/521,565, filed on May 25, 2004.

(51) Int. Cl. A61K 8/00

(52) U.S. Cl. 510/130

(57) ABSTRACT

The invention described herein includes a formulation comprising: a lower than expected use level of structurant/surfactant blend and an emulsion comprising a homogenized mixture of wax and alcohol components, at least one of which is a surfactant, wherein the formulation comprises a stable lamellar or spherulite phase.

### RELATED APPLICATIONS

This application is a continuation-in-part under 37 C.F.R. 1.53(b) of U.S. Ser. No. 10/710,052 filed Jun. 15, 2004, which claims the benefit of U.S. Provisional Application Ser. No. 60/521,565 filed May 25, 2004, which applications are incorporated herein by reference.

### TECHNICAL FIELD

Invention embodiments described herein relate to personal care products that cleanse and moisturize skin of a user through foaming action, as well as a method of making the personal care product, and a method of using the personal care product.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the software and data as described below and in the drawings that form a part of this document: Copyright 2005, Coty, Inc. All Rights Reserved.

### BACKGROUND ART

Personal care products have a variety of applications, including topical application to skin. The topical applications have acted to moisturize, cleanse, disinfect or to apply active agents to the skin. Typically, the topical applications have performed only one of these functions. One type of personal care product, a product that cleanses skin has, in some instances, been used as a structurant in order to produce a composition having a lamellar or spherulite phase.

It has been reported that formation of lamellar dispersion based compositions can only be accomplished with a limited, small group of surfactants. Surfactants not falling within this small group have been reported to crystallize out of solution when added to a composition containing a structurant, or to destabilize the composition or both. The WO 97/05857 patent application includes a table that describes performances of a collection of test surfactants in forming a lamellar phase when added to a composition containing a structurant. The data in the table stated that stearyl alcohol, glyceryl monostearate and cetyl alcohol did not form lamellar phases at room temperature. Instead, these materials crystallized out of solution or destabilized the lamellar phase formed by the composition.

### DISCLOSURE

Embodiments of the invention described herein include a formulation and system for cleansing and moisturizing skin, wherein the formulation and system include a stable, aqueous dispersion of cleansing and moisturizing agents that are structured within a stable, spheroidal network of finely divided cleansing and moisturizing particles. The stable, spheroidal network is capable of foaming due to flocculation in water and mechanical action by a consumer, which, in one embodiment, occurs in the shower. The spheroidal network also includes wetting agents and emulsifiers such as stearic acid, cetyl alcohol, glyceryl monostearate and stearyl alcohol, that are incorporated within the network. The wetting agents and emulsifiers are desirable because they aid in building viscosity of the formulation, and aid in producing a high yield value. Further, the wetting agents and emulsifiers aid in skin occlusiveness for increased moisturization. Heretofore, it has not been thought possible to make a stable, spheroidal network of finely divided cleansing and moisturizing particles using a surfactant in a concentration of less than 30.00 percent by weight. It has surprisingly been found that the stable, spherical network can be made using a surfactant concentration that is less than about 30.00 percent by weight. In one embodiment, a stable, spherical network was made using a surfactant concentration of about 26.00 by weight.

A cleansing-foaming lotion having the reduced surfactant concentration is, in one embodiment, made by obtaining or making the following base concentrate:

| | Grams |
|---|---|
| Phase A | |
| DI Water | 7.000 |
| Guar hydroxypropyltrimonium chloride | 0.500 |
| EDTA disodium salt | 0.070 |
| Glycerine 99% USP | 1.000 |
| Cyamposis Tetragonoloba (Guar) Gum | 0.300 |
| Structurant Blend | 30.000 |
| Uvinul MS 40 Powder | 0.200 |
| Grape Seed Oil | 2.000 |
| Silicone 200/500 | 0.750 |
| Ethyl Hexyl Hydroxystearate | 1.000 |
| C12-15 Alkyl Ethyl Hexanoate | 0.750 |
| Stearic Acid Tri Press | 0.500 |
| Cetyl Alcohol | 0.500 |
| Glyceryl Monostearate | 0.250 |
| Stearyl Alcohol | 0.500 |
| Petrolatum | 6.000 |
| Shea Butter | 3.000 |
| Preservative | 1.000 |
| Phase B | |
| Extract | |
| Additives | |
| Fragrance | |
| Phase C | |
| DI Water | |
| Deionized Water is added in a quantity to take the weight of 79 | |
| percent of the weight of phase A to about 100 gms. | |
| Phase D | |
| NaCl (Neat) | 5.000 |
| Citric Acid 20% Aq. Soln. | 1.000 |

The ingredients in Phase A are combined in the proportions described to make a preparation having a weight that is 79.00 percent of the weight of the original base concentrate described in the Phase A formulation described above. In one embodiment, the base concentrate is made using the quantities shown above for Phase A. The base concentrate is then weighed. A quantity of 79.00 percent of the weighed base concentrate is obtained for further processing. Extract, additives and fragrance are then added to the 79.00 percent preparation. These ingredients are shown as Phase B. Next, water is added in a quantity shown in Phase C and ingredients in Phase D are added.

The deionized water is added to form a cleansing foaming lotion having a volume of about 100 milliliters. With this dilution, the surfactant concentration is about 26 percent.

This formulation may be scaled up or down. It was not thought possible heretofore to make a stable formulation with spherulites having the lower ingredient concentrations described herein.

While specific types of wetting agents and emulsifiers are described herein, it is understood that embodiments of the invention described herein are not limited to the specific wetting agents and emulsifiers described.

Embodiments of the invention additionally include methods for making the formulation and system, as well as methods for adding wetting agents and emulsifiers to a structurant in a manner that prevents the wetting agents and emulsifiers from “salting out.” Furthermore, the wetting agents and emulsifiers such as stearic acid, cetyl alcohol, glyceryl monostearate and stearyl alcohol do not destabilize the activity of the structurant. Embodiments of the invention also include methods for using the formulation and system of the invention.

Prior to embodiments of the invention described herein, it has not been thought possible to incorporate wetting agents and emulsifiers such as stearic acid, cetyl alcohol, glyceryl monostearate, and stearyl alcohol into a formulation with a structurant to make a spheroidal network. Embodiments of the invention described herein include stearic acid, cetyl alcohol, glyceryl monostearate and stearyl alcohol as well as a structurant to make the spheroidal network. It has surprisingly been found that the stearic acid, cetyl alcohol, glyceryl monostearate, and stearyl alcohol do not salt out of the network and do not destabilize the network but are incorporated into the network without introducing instability. To the contrary, it has unexpectedly been found that these wetting agents aid in building viscosity of the spheroidal network and aid in producing a high yield value. While stearic acid, cetyl alcohol, glyceryl monostearate, and stearyl alcohol are described, it is understood that other alcohols and waxes are suitable for use in the stable formulation of the invention.

It has also surprisingly been found that a stable spheroidal network can be formed with a reduced amount of surfactant. This new formulation is formed by reducing the amount of surfactant from the concentrated formula described above and adding water so that the surfactant concentration is about 26 percent. With this method, a stable, spheroidal network can be prepared with less than 30.00 percent surfactant.

As used herein, the term “lamellar” refers to an ordered liquid crystalline phase having alternating surfactant bilayers and water layers.

The term “spheroidal network” as used herein refers to a lamellar phase that conforms to form a submicron spherical onion. Vesicles and liposomes are types of spheroidal networks.

The term “structurant” as used herein refers to a molecule that aids in the formation of a spheroidal network.

The term “lotion” refers to a cosmetic formulation applied to the skin.

The term “wash” refers to a cosmetic formulation that is applied to the skin and then is washed from the skin.

The spheroidal network included in invention embodiments described herein has a multilayer structure conformed to a submicron onion shape. Insoluble materials are dispersed throughout the onion shaped spheroidal network. For some embodiments, insoluble materials are enclosed within the interior layers of the spheroidal network as well as the outer layers. Soluble materials are similarly dispersed throughout the spheroidal network, within interior layers and outer layers.

A structurant is a component of a cleansing-moisturizing wash of the invention. Compositions of embodiments of the invention that employ structurants have, in some embodiments, lamellar or spherulitic phases that are capable of suspending large particles within the phase while remaining pourable. Structurants are also used to prepare product embodiments of the invention that impart a soft feel that is pleasing to consumers. One type of structurant is an electrolyte-based structurant. Examples of electrolyte-based structurants usable in the formulation and system embodiments of the invention described herein are described in the Coty application Ser. No. 10/710,052, publication no. 2005/0025731A1 . While specific electrolyte-based structurants are described herein, it is believed that other electrolyte-based structurants are suitable for use in embodiments of the invention.

One cleansing-moisturizing wash embodiment of the invention has foaming functionality that aids in the cleansing functionality. This embodiment of the cleansing-moisturizing wash includes four phases. Ingredients in the four phases for one embodiment are shown in the table that follows. It is understood that this embodiment is presented as one example of the invention described herein and is not presented to limit the invention.

| | gms |
|---|---|
| Phase A | |
| DI Water | 5.530 |
| Guar hydroxypropyltrimonium chloride | 0.395 |
| Water | |
| EDTA disodium salt | 0.0553 |
| Glycerine 99% USP | 0.790 |
| Cyamposis Tetragonoloba (Guar) Gum | 0.237 |
| Miracare SLB-365 Surfactant Blend | 26.070 |
| Sodium Laureth Sulfate | 3.950 |
| DI Water | 24.0002 |
| Trilon BD | 0.0553 |
| Grape Seed Oil | 1.58 |
| Silicone 200/50 | 0.5925 |
| Ethyl Hexyl Hydroxystearate | 0.79 |
| C12-15 Alkyl Ethyl Hexanoate | 0.5925 |
| Stearic Acid Tri Press | 0.395 |
| Cetyl Alcohol | 0.395 |
| Glycerol Monostearate | 0.1975 |
| Steryl Alcohol | 0.395 |
| Petrolatum | 4.74 |
| Shea Butter | 2.37 |
| Preservative | 0.79 |
| Sodium Chloride (Neat) | 3.95 |
| Cirtric Acide 20% Aqueous Solution | 1.185 |
| Phase B | |
| Deionized Water is added in a quantity to take the weight of | |
| phase A to about 100 gms. | |
| Phase C | |
| NaCl (Neat) | 1.000 |
| Citric Acid 20% Aq. Soln. | 0.400 |
| Phase D | |
| Color as needed | |

A first phase, phase A, of the wash includes a cleansing foaming lotion base that includes a cationic conditioning agent, guar hydroxypropyltrimonium chloride, and a chelating agent, EDTA disodium salt and other non-toxic salts. Other cation conditioning polymers which are suitable for use in phase A, include Polyquaternium-4, Polyquaternium-6, Polyquaternium-7, Polyquaternium-10, Polyquaternium-11, Polyquaternium-16, Polyquaternium-24, and Polyquaternium-39. While specific quantities of ingredients are described in the table, it is understood that other concentration ranges may be suitable for use in formulation embodiments of the invention. The cationic conditioning range may be from 0.001 to 1.000 percent by weight. The chelating agent range may be from 0.001 to 0.250 by weight.

Phase A also includes glycerine, the surfactant blend in a concentration of about 30 to 40 percent, and the Cyamopsis Tetragonoloba (Guar) Gum. In one embodiment, the surfactant blend is Sodium Lauroamphoacetate, Sodium Trideceth Sulfate and Cocamide MEA.

Phase A further includes lotion ingredients such as grape seed oil, silicones, esters, wetting agents and emulsifiers such as stearic acid tri press, cetyl alcohol, glycerol monostearate, steryl alcohol, sodium lauryl sulphate, fatty alcohol, ether sulfates, disodium-n-lauryl-β-imino dipropionate, polyoxyethylinized castor oil, or sorbitan monooleate, sorbitan monostearate, lecithin, polyoxyethylene stearate, alkyl phenol polyglycol ether, cetyltrimethyl ammonium chloride, or mono-/dialkylpolyglycol ether-orthophosphorus acid-mono-ethanolamine salts, petrolatum and shea butter. Other ingredients are suitable for use in phase A to formulate a lotion.

Phase B includes deionized water. The deionized water is added to take the formulation to 100 milliliters. At this concentration of deionized water, the surfactant blend has a concentration of about 26.00 percent of the formulation. This concentration of surfactant is much lower than the concentration of surfactant described in conventional spherulite containing formulations.

The concentration range for use of sodium chloride in Phase C ranges from 1.000 to 6.000 percent by weight. The concentration range for use of citric acid ranges from 0.001 to 3.000 percent by weight. If required, the pH is adjustable with sodium hydroxide or any other pH adjusting electrolyte, also within a range of 0.001 to 3.000 percent.

Some embodiments of Phase A include a preservative. In one embodiment, the preservative is Phenonip. Other preservatives suitable for use include phenoxyethanol, parabens, DMDM hydantoin, chlorophenesin, benzyl alcohol, chlorhexidine gluconate, an ethyl alcohol containing pentylene glycol and a sodium methylparaben mixture in the proportions 47/47/6, a pentylene glycol and sodium methylparaben mixture, Methylchloroisothiazolinone, Methylisothiazolinone, and mixtures thereof in a concentration range of 0.001 to 1.000 percent by weight, based upon proven efficacy per formula embodiment.

In addition to the above noted compounds, various other ingredients can optionally be utilized in the stable composition of the present invention such as Fragrances, Perfumes, Preservatives, Disinfectants, Deodorizers, Antiperspirants, Antioxidants, Antiredeposition Agents, Carriers, Chelating and Sequestering Agents, Dyes and Pigments, Quaternary Conditioners, Cationic conditioning polymers such as, Polyquaternium-4, Polyquaternium-6, Polyquaternium-7, Polyquaternium-10, Polyquaternium-11, Polyquaternium-16, Polyquaternium-24, and Polyquaternium-39, Corrosion Inhibitors, Hydrotropes, Coupling Agents, Defoamers, Builders, Dispersants, Emollients, Extracts, Vitamins, Enzymes, Foam Boosters, Flocculants, Whitening Agents, Fixative Polymers such as PVP, Humectants, Opacifiers, Plasticizers, Powders, Solubilizers, Solvents, Waxes, UV Absorbers/UV Light Stabilizers, Hydrolyzed Proteins, Keratin, Collagens, and the like. Fragrance ingredients are not added to Phase A.

In the formulation embodiment of the table described above, Phase A was prepared by mixing the guar hydroxypropyltrimonium chloride with water and EDTA disodium. The Cyamopsis Tetragonoloba (Guar) Gum and glycerine were blended until the Cyamopsis Tetragonoloba (Guar) Gum was wetted and was completely dispersed to make a slurry. The glycerine/Cyamopsis Tetragonoloba (Guar) Gum slurry was added to the surfactant blend and was mixed until a uniform blend was achieved.

The two premixes were mixed until uniform and then heated to a temperature within a range of 40 degrees C. to 70 degrees C. Separately the following ingredients were blended: Grape seed oil, silicone 200/500, EthylHexyl Hydorstearate, C12-15 Alkyl EthylHexanoate, stearic acid tri pres, cetyl alcohol, glycerol monostearate, stearyl alcohol, petrolatum, shea butter and deionized water. This ingredient mixture was heated until all ingredients were liquid. When all ingredients were liquid, the mixture was mixed at high energy and at moderate speed. The moderate speed mixing was continued until the mixture was ready to add to the combined mixture above, also called the main batch. The mixture was cooled to room temperature at a rate of 1 degree Centigrade for every 10 minutes. Phase A was blended to form a structured surfactant system. The structured surfactant system formed spherulites. Once spherulites in phase A were formed, additional water was added to dilute the system to about 26.00% of the surfactant blend.

Phase B was then added to make a combined blend of phase A and B. The mixture of phase A and phase B was mixed until a homogeneous mixture was achieved. Phase C and Phase D were both added to the batch, respectively, mixing each for fifteen minutes minimum between ingredient additions.

The cleansing foaming lotion embodiments of the invention described herein are viscous liquids, having a viscosity of about 100,000 cPs and a pH of 5.5 to 6.5. The wash embodiments, are, for some embodiments, colored or fragranced. Some embodiments of the cleansing foaming lotion may moisturize skin for at least about 24 hours from application.

One lotion embodiment of the invention foams and cleanses for use in the shower and may moisturize skin for at least about 24 hours after showering. With this embodiment, a user need use only a product of the invention to both cleanse and moisturize. A separate cleanser and moisturizer are not required. That the invention described herein is capable of both cleansing and moisturizing is unexpected because cleansing skin removes fats, oils and lipids from the skin. Cleansing typically leaves skin dehydrated. It is then unexpected that a single product is capable of both cleansing and moisturizing.

One additional attribute of the wash of the invention described herein is that the cleansing moisturizing wash moisturizes skin without leaving a heavy oil feel on the skin. This heavy moisturized feel typically occurs when a moisturizer is separately applied to skin. The wash described herein not only saves a user time and money in not having to apply two separate products but eliminates the heavy oil feel while effectively moisturizing.

The cleansing foaming wash of the invention described herein differs from a traditional wash in that the traditional wash does not include a spherulite state. This spherulite state is also known as an “onion phase” state. The spherulite state is a stable high energy state. The spherulites within the wash make it possible to add lotion ingredients to a cleansing and foaming product and to maintain all of the properties of both the body wash, which cleanses and the lotion, which moisturizes.

The cleansing foaming wash with a lotion is a structured liquid formulation that includes water soluble, water dispersible, water insoluble and water indispersible ingredients without an adverse impact, such as “salting out” and incipient instability. The cleansing portion of the formulation also includes adjuvants and solubilizers that aid in creating a product with a pre-selected viscosity or foaming potential. The formulation of the invention described herein produces a finished product that has improved stability as compared to conventional washes because of materials in the formulation, that add stability, and that cannot be added to a conventional wash. These materials include wetting agents and emulsifiers such as stearic acid, cetyl alcohol, glyceryl monostearate and stearyl alcohol.

A method for making a formulation embodiment of the invention that includes waxes and alcohols such as stearic acid, cetyl alcohol, glyceryl monostearate and stearyl alcohol, or other wax or alcohol-based surfactant, includes the steps described herein. One embodiment of Phase A, such as is described above, was prepared by adding oils, silicones, esters and any other insoluble ingredients to the alcohols and waxes. About 10 percent deionized water was also added.

Phase A was heated to about 70 degrees Centigrade to melt the alcohols and waxes. When all of the alcohols and waxes were melted the phase A oil phase was emulsified and homogenized using, in one embodiment, a homo-mixer at moderate speed. Phase A was blended and mixed for a minimum of fifteen minutes. Moderate sidesweep mixing was continued until the mixture was mixed homogeneously. An addition of sodium chloride and/or citric acid drove the formula to a spherulite phase. It was observed that the spherulite phase was stable with no salting out.

One test for determining whether the formulation is in a spherulite phase includes taking a 100 gram formulation sample of the formulation and adding 1 to 2 percent neat salt. If, at 24 hours the viscosity has increased above the initial viscosity of the batch, the formulation is not in the spherulite phase. This result indicates that the formulation requires more oils or salt in order to form a spherulite phase. A second test includes preparing a sample of about 100 grams and adding several beads to the sample. The sample is held at a temperature of about 50 degrees Centigrade for several days. If the beads are still suspended, the sample has a spherulite phase and is stable.

Embodiments of the formulation of the invention described herein have use in shaving and moisturizing, shampoos and conditioners, in addition to washes and moisturizers. Formulation embodiments of the invention described herein have a wide number of other applications such as personal care applications, home care applications, industrial and institutional applications, pharmaceutical applications, textile compounds, and the like.

Examples of various personal care applications include products such as the following: Shampoos, for example Baby Shampoos; Conditioning Shampoos; Bodifying Shampoos; Moisturizing Shampoos; Temporary Hair Color Shampoos; 3-in-1 Shampoos; Anti-Dandruff Shampoos; Hair Color Maintenance Shampoos; Acid (Neutralizing) Shampoos; Salicylic Acid Shampoos;

Skin and Body Cleansers, for example Moisturizing Body Washes; Antibacterial Body Washes; Deodorizing Body Washes; Bath Gels; Shower Gels; Hand Soaps; Bar Soaps; Body Scrubs; Bubble Baths; Facial Scrubs; Foot Scrubs; Creams and Lotions, for example Alpha-Hydroxy Acid Washes, Lotions and Creams; Beta-Hydroxy Acid Washes, Creams and Lotions; Skin Whiteners; Self Tanning Washes and Lotions; Sunscreen Washes and Lotions; Barrier Lotions; Moisturizers; Hair Styling Creams; Vitamin C Creams; Liquid Talc Products and Antibacterial Lotions; and other moisturizing lotions and creams;

Skin and Hair Gels, for example Facial Masks; Body Masks; Hydroalcoholic Gels; Hair Gels; Body Gels; Sunscreen Gels; and the like, as well as other personal care applications such as permanent hair color, and the like.

Examples of home care applications include products such as home care and industrial and institutional applications, such as laundry detergents; dishwashing detergents (automatic and manual); hard surface cleaners; hand soaps, cleaners and sanitizers; polishes (shoe, furniture, metal, etc.); automotive waxes, polishes, protectants, and cleaners, and the like.

Examples of pharmaceutical applications include topical formulations in the form of creams, lotions, ointments, or gels, wherein the formulation may be used as a carrier for the pharmaceutically active material, or as a carrier for a skin penetration enhancer, or as a carrier for a phase having an aesthetic effect, or present to enhance the solubility or bioavailability of the pharmaceutically active material.

These formulations may be administered or applied to either human or veterinary conditions for the full breadth of indications treatable by pharmaceutical means, such as fever, irritation, dermatitis, rash; viral, fungal, or bacterial infection; organic disease; etc. The pharmaceutically active agents could have any appropriate function for treatment of the condition, and can be a mixture of one or more pharmaceutically active materials, such as emetics, antiemetics, febrifuge, fungicide, biocide, bactericide, antibiotic, antipyretic, NSAID, emollient, analgesics, antineoplastics, cardiovascular agents, enzymes, proteins, hormones, steroids, antipruritics, antirheumatic agents, biologicals, cough and cold treatments, dandruff products, muscle relaxants, psychotherapeutic agents, skin and mucous membrane agents, skin care products, vaginal preparations, wound care agents, and other appropriate classes of pharmaceutically active agents capable of appropriate administration via dosage form.

The formulation embodiments may be packaged in a pressurized container or unpressurized container. The formulation may be applied to wipes, swabs or other flexible substrates.

The formulation embodiments may include variegation, and suspended solids that impart color. The formulation embodiments may be made into a wide variety of product types that include, but are not limited to, lotions, creams, gels, sticks, sprays, ointments, cleansing liquid washes, solid bars, shampoos, pastes, foams, powders, mousses, shaving creams, wipes, patches, nail lacquers, wound dressing, adhesive bandages, hydrogels, and films. Make-up, such as foundations, mascaras, and lipsticks also form suitable compositions. These product embodiments may further comprise several additional types of cosmetically acceptable topical carriers including, but not limited to solutions, emulsions (e.g., microemulsions and nanoemulsions), gels, solids and liposomes.

While certain embodiments of the present invention have been described and specifically exemplified above, it is not intended that the invention be limited to such embodiments. Various modifications may be made thereto without departing from the scope and spirit of the present invention, as set forth in the following claims.

1. A formulation comprising: a structured surfactant system comprised of a surfactant in a concentration of less than 30 percent by weight, wherein the formulation comprises a stable lamellar or spherulite phase.

2. The formulation of claim 1 wherein the formulation is comprised of an emulsion, wherein the formulation comprises a stable lamellar or spherulite phase.

3. The formulation of claim 1 wherein the structurant comprises an electrolyte.

4. The formulation of claim 1, wherein the emulsion further comprises grape seed oil.

5. The formulation of claim 1, wherein the emulsion further comprises one or more silicone.

6. The formulation of claim 1, wherein the emulsion further comprises one or more ester.

7. The formulation of claim 1, wherein the emulsion further comprises water.

8. The formulation of claim 1 further comprising one or more ingredients that moisturize skin.

9. The formulation of claim 1 further comprising one or more ingredients that cleanse skin.

10. The formulation of claim 1, further comprising one or more ingredients that cleanse and moisturize skin.

11. An after-shave comprising the formulation of claim 1.

12. A shower wash comprising the formulation of claim 1.

13. A shampoo comprising the formulation of claim 1.

14. A wipe comprising the formulation of claim 1.

15. A swab comprising the formulation of claim 1.

16. A pharmaceutical comprising the formulation of claim 1.

17. A variegated cosmetic comprising the formulation of claim 1.
